SEQUENCE LISTING (4 sequences)

SEQ ID NO: 1 - DNA, 11 bp, Homo sapiens
ggggacgcgga

SEQ ID NO: 2 - DNA, 11 bp, Hiomo sapiens
agggcaaagga

SEQ ID NO: 3 - DNA, 11 bp, Homo sapiens
aagaacgagga

SEQ ID NO: 4 - DNA, 11 bp, Homo sapiens
gggaacgcgaa

(19) United States
(12) Patent Application Publication
(10) Pub. No.: US 2012/0288858 A1
(43) Pub. Date: Nov. 15, 2012

(54) ASSESSING SMALL CELL LUNG CANCER OUTCOMES

(76) Inventors: Ping Yang, Rochester, MN (US); Zhifu Sun, Rochester, MN (US)

(21) Appl. No.: 13/463,486

(22) Filed: May 3, 2012

Related U.S. Application Data
(60) Provisional application No. 61/482,020, filed on May 3, 2011.

(51) Int. Cl. C12Q 1/68

(52) U.S. Cl. 435/6.11

(57) ABSTRACT

This document provides methods and materials involved in assessing lung cancer (e.g., SCLC). For example, methods and materials for identifying a mammal having lung cancer (e.g., SCLC) as being susceptible to a poor outcome are provided.

### CROSS-REFERENCE TO RELATED APPLICATIONS

This application claims the benefit of U.S. Provisional Application Ser. No. 61/482,020, filed May 3, 2011. The disclosure of the prior application is considered part of (and is incorporated by reference in) the disclosure of this application.

### STATEMENT AS TO FEDERALLY SPONSORED RESEARCH

This invention was made with government support under grant numbers CA077118, CA080127, and CA084354 awarded by the National Institutes of Health. The government has certain rights in the invention.

### BACKGROUND

1. Technical Field

This document relates to methods and materials involved in assessing lung cancer (e.g., small cell lung cancer). For example, this document provides methods and materials for determining whether or not a mammal having lung cancer (e.g., small cell lung cancer) is susceptible to a poor outcome.

2. Background Information

Small cell lung cancer (SCLC) is the most aggressive cell type among lung cancer subtypes with a median survival following diagnosis being estimated to be 8-20 months. Virtually all patients with SCLC are treated with chemotherapy and/or radiation therapy. Platinum-containing compounds (e.g., cisplatin and carboplatin) are most commonly used, and patients' survivals vary substantially.

### SUMMARY

This document provides methods and materials involved in assessing lung cancer (e.g., SCLC). For example, this document provides methods and materials for identifying a mammal having lung cancer (e.g., SCLC) as being susceptible to a poor outcome. As described herein, the presence of one or more genetic variations in the glutathione synthetase (GSS) gene, one or more genetic variations in the ATP-binding cassette, sub-family C, member 2 (ABCC2) gene, or one or more genetic variations in the X-ray repair cross-complementing protein 1 (XRCC1) gene can indicate that a person with lung cancer (e.g., SCLC) has an increased susceptible to a poor outcome (e.g., death within one, two, three, or four years). In some cases, an allele having a genetic variation in the GSS, ABCC2, or XRCC1 gene that is associated to an increased risk of death from lung cancer can be referred to as a risk allele, and the presence of multiple risk alleles (e.g., 2, 3, 4, or 5 risk alleles) can indicate that the person with lung cancer (e.g., SCLC) has an increased susceptible to a poor outcome (e.g., death within one, two, three, or four years) as compared to a person with lung cancer (e.g., SCLC) having only one risk allele. Identifying lung cancer patients who have a poor prognosis can allow such patients to be offered more aggressive therapy earlier.

In general, one aspect of this document features a method for assessing lung cancer. The method comprises, or consists essentially of, (a) detecting the presence of a genetic variation in ABCC2 nucleic acid, (b) detecting the presence of a genetic variation in GSS or XRCC1 nucleic acid, and (c) classifying the mammal as being susceptible to a poor lung cancer outcome based at least in part on the presence of the genetic variation in ABCC2 nucleic acid and the presence of the genetic variation in GSS or XRCC1 nucleic acid. The mammal can be a human. The genetic variation in ABCC2 nucleic acid can be rs11597282. The method can comprise detecting the presence of a genetic variation in GSS nucleic acid. The genetic variation in GSS nucleic acid can be rs2025096, rs7265992, or rs6060127. The method can comprise detecting the presence of a genetic variation in XRCC1 nucleic acid. The genetic variation in XRCC1 nucleic acid can be rs2854510 or rs1001581. The poor lung cancer outcome can comprise death within two years of diagnosis of lung cancer. The poor lung cancer outcome can comprise death within four years of diagnosis of lung cancer. The lung cancer can be small cell lung cancer.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a linkage disequilibrium (LD) structure plot for the glutathione synthetase (GSS) gene. SNPs typed are indicated by arrows. Solid arrows show significant association with survival of SCLC, while dashed arrows are not significant. Numbers in the plot are LD r². The boxes with dark color have strong linkage, while the lighter ones are associated with weak LD. Two LD blocks were defined.

FIG. 2 is a LD plot for the ABCC2 gene. SNPs typed are indicated by arrows. Solid arrows show significant association with survival of SCLC, while dashed arrows are not significant. Numbers in the plot are LD r². The boxes with dark color have strong linkage, while the lighter ones are associated with weak LD.

FIG. 3 is a LD plot for the XRCC1 gene. SNPs typed are indicated by arrows. Solid arrows show significant association with survival of SCLC, while dashed arrows are not significant. Numbers in the plot are LD r².

FIG. 4 is a Kaplan-Meier survival graph plotting the percent survival for patients carrying 1, 2, 3, or 4 risk alleles across three top SNPs from gene ABCC2 (rs11597282), GSS (rs2025096), and XRCC1 (rs1001581). The highest number of the risk alleles observed in the evaluated population was 5. Because of the small number, the patients with 5 risk alleles were combined with the group having 4 risk alleles. The p-value was obtained from the log rank test.

### DETAILED DESCRIPTION

This document provides methods and materials related to assessing lung cancer in mammals. For example, this document provides methods and materials for identifying lung cancer patients as having a high level of susceptibility to a poor lung cancer outcome by determining whether or not the patient contains one or more genetic variations in the GSS gene, one or more genetic variations in the ABCC2 gene, and/or one or more genetic variations in the XRCC1 gene. As described herein, the presence of one or more genetic variations in the GSS gene, one or more genetic variations in the ABCC2 gene, and/or one or more genetic variations in the XRCC1 gene can indicate that the lung cancer (e.g., SCLC) patient has an increased susceptible to a poor outcome (e.g., death within one, two, three, or four years). In some cases, the presence of multiple risk alleles (e.g., 2, 3, 4, or 5 risk alleles) can indicate that the lung cancer (e.g., SCLC) patient has an increased susceptible to a poor outcome (e.g., death within one, two, three, or four years) as compared to a lung cancer (e.g., SCLC) patient having only one GSS, ABCC2, or XRCC1 risk allele.

An example of a human GSS nucleic acid can have the sequence set forth in GenBank® GI No. 30581166. Human ABCC2 nucleic acid can have the sequence set forth in GenBank® GI No. 188595701. Human XRCC1 nucleic acid can have the sequence set forth in GenBank® GI No. 190684674. Examples of genetic variations of a GSS gene that are associated with an increased risk of death from lung cancer include, without limitation, rs7265992 and rs6060127. The presence of any one or more of these genetic variations (minor alleles) for a GSS allele can indicate that that GSS allele is a risk allele. An example of a genetic variation of an ABCC2 gene that is associated with an increased risk of death from lung cancer includes, without limitation, rs11597282. The presence of this genetic variation (minor allele) for an ABCC2 allele can indicate that that ABCC2 allele is a risk allele. Examples of genetic variations of an XRCC1 gene that are associated with an increased risk of death from lung cancer include, without limitation, rs2854510 and rs1001581. The presence of any one or more of these genetic variations (minor alleles) for an XRCC1 allele can indicate that that XRCC1 allele is a risk allele.

In some case, the presence of minor alleles in rs2025096, rs2236270, and/or rs2273684 (GSS; A, A, and C, respectively) can indicate that the patient has a reduced risk of death from lung cancer.

The presence or absence of a genetic variation in GSS, ABCC2, or XRCC1 nucleic acid can be determined using any appropriate technique. For example, nucleic acid sequencing techniques, PCR-based techniques, and nucleic acid-based mutation detection techniques can be performed to detect the presence or absence of a genetic variation in GSS, ABCC2, or XRCC1 nucleic acid.

This document also provides methods and materials to assist medical or research professionals in identifying a mammal as being susceptible to a favorable or poor lung cancer outcome. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining whether or not a patient contains one or more genetic variations in the GSS gene, one or more genetic variations in the ABCC2 gene, and/or one or more genetic variations in the XRCC1 gene and (3) communicating information about that patient's GSS, ABCC2, and/or XRCC1 genes to that professional. In some cases, a professional can be assisted by (1) determining the number of GSS, ABCC2, and/or XRCC1 risk alleles of a patient and (3) communicating information about that patient's number of GSS, ABCC2, and/or XRCC1 risk alleles to that professional.

Any method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

The invention will be further described in the following examples, which do not limit the scope of the invention described in the claims.

### EXAMPLES

### Example 1

### Genetic Variation in Glutathione Metabolism and DNA Repair Genes Predicts Survival of Small Cell Lung Cancer Patients

### Subjects

The participants in this example were diagnosed SCLC lung cancer patients who consented to the research protocol as approved by an institutional review board. Detailed descriptions of identification, enrollment, blood collection, and follow-up were described elsewhere (Sun et al., J. Thoracic Cardiovascular Surgery, 131:1014-1020 (2006) and Yang et al., Chest, 128:452-462 (2005)). Briefly, each case was identified through the Mayo Clinic pathologic diagnostic system, their medical records were abstracted, and blood samples were collected. Tumor stage was defined as limited stage (involving one lung or with lymph node involvement on the same side of the chest) and extensive stage where cancer has spread to the other lung, to lymph nodes on the other side of the chest, or to distant organs. Smoking information was obtained from medical record abstraction, questionnaires, and/or patient interviews. Pack-years were calculated by multiplying the number of packs per day by the total years of smoking Never smokers were defined as having had smoked fewer than 100 cigarettes during their lifetime, and former smokers were defined as having had at least six months of smoking abstinence at the time of diagnosis. Vital status and cause of death were determined by reviewing the Mayo Clinic registration database and medical records, correspondence from patients' next-of-kin, death certificates, obituary documents, the Mayo Clinic Tumor Registry, and the Social Security Death Index website. Additional patient information was collected with a mail-in questionnaire sent to participants or to their next-of-kin annually until patient's death.

### Gene and SNP Selection

Genes in the glutathione pathway were included as described elsewhere (Yang et al., J. Clin. Oncol., 24:1761-1769 (2006)). Twenty-nine genes, including isozymes and membrane bound transporter proteins, were selected. An additional 20 genes were selected from the DNA repair pathway following a review of the literature that reported an association with treatment response or survival in lung or other cancers (Table 1). Tag-SNPs on these genes were selected based on HapMap data (Release 22/Phase II on NCBI B36) by Haploview, Version 3 (http://www.broad.mit.edu/mpg/haploview/), using the Caucasian (CEU) data available from HapMap (http://www.hapmap.org/). Tag-SNP selection parameters ignored pairwise comparisons of markers greater than 500 kb apart; excluded individuals with greater than 50% missing genotypes; excluded SNPs with Hardy-Weinberg p-values of less than 0.001, SNPs with fewer than 75% genotype calls, SNPs with more than one Mendelian error, and SNPs with a minor allele frequency less than 0.001; performed aggressive tagging using a r² threshold of 0.8, and included a LOD threshold for multi-marker tests of three. The genes, genotyped SNPs, and the SNPs in final analysis after quality assessment are presented in Table 1.

TABLE 1
Genotyped SNPs on the genes in glutathione
and DNA repair pathways.

| | | | Number | Number in |
|---|---|---|---|---|
| Pathway | Gene | Chr | tested | Analysis |
| GSH | ABCC1 | 16p13.1 | 32 | 31 |
| | ABCC2 | 10q24 | 12 | 11 |
| | ABCC3 | 17q21 | 14 | 14 |
| | ABCC4 | 13q31 | 68 | 64 |
| | GCLC | 6p12 | 16 | 14 |
| | GCLM | 1p21 | 3 | 3 |
| | GPX1 | 3p21.3 | 3 | 1 |
| | GPX2 | 14q24.1 | 3 | 3 |
| | GPX3 | 5q23 | 6 | 6 |
| | GPX5 | 6p22.1 | 3 | 3 |
| | GPX6 | 6p22.1 | 3 | 3 |
| | GPX7 | 1p32 | 2 | 2 |
| | GSR | 8p21.1 | 7 | 7 |
| | GSS | 20q1.2 | 7 | 7 |
| | GSTA1 | 6p12 | 3 | 2 |
| | GSTA2 | 6p12.2 | 4 | 2 |
| | GSTA3 | 6p12 | 5 | 5 |
| | GSTA4 | 6p12 | 9 | 7 |
| | GSTA5 | 6p12.1 | 6 | 5 |
| | GSTM1 | 1p13.3 | 2 | 0 |
| | GSTM2 | 1p13.3 | 1 | 1 |
| | GSTM3 | 1p13.3 | 3 | 3 |
| | GSTM4 | 1p13 | 4 | 4 |
| | GSTM5 | 1p13.3 | 7 | 5 |
| | GSTO1 | 10q25.1 | 8 | 5 |
| | GSTO2 | 10q25.1 | 10 | 6 |
| | GSTP1 | 11q13-qter | 9 | 5 |
| | GSTT1 | 22q11.23 | 7 | 2 |
| | GSTZ1 | 14q24.3 | 10 | 9 |
| DNA Repair | APEX1 | 14q11.2 | 3 | 2 |
| | ERCC1 | 19q13.32 | 5 | 5 |
| | ERCC2 | 19q13.3 | 7 | 7 |
| | LIG1 | 19 | 5 | 5 |
| | MGMT | 10q26 | 42 | 38 |
| | MLH1 | 3p22.3 | 2 | 2 |
| | MSH2 | 2p21 | 6 | 6 |
| | MSH3 | 5q11-q12 | 16 | 16 |
| | MSH6 | 2p16 | 9 | 9 |
| | OGG1 | 3p26 | 4 | 3 |
| | PARP | 1q41-q42 | 13 | 12 |
| | PCNA | 20pter-p12 | 1 | 1 |
| | POLB | 8p12-p11 | 1 | 1 |
| | RAD50 | 5q23-q31 | 3 | 3 |
| | RAD51 | 15q15.1 | 3 | 3 |
| | RAD52 | 12p13-p12.2 | 10 | 10 |
| | RRM1 | 11p15.5 | 6 | 6 |
| | XPA | 9q22.3 | 5 | 5 |
| | XPC | 3p25 | 6 | 6 |
| | XRCC1 | 19q13.2 | 5 | 5 |
| Total | | | 419 | 375 |

### Genotyping and Quality Control

Four hundred and nineteen tag-SNPs (267 from the glutathione and 152 from DNA repair pathways) were genotyped in the Mayo Clinic Genomic Shared Resources using a custom-designed Illumina GoldenGate panel. Intensity data were imported into BeadStudio software for clustering and review. All samples were successfully genotyped, with an average call rate of 99.1 percent. For the SNPs, 95.2% (399/419) were successfully genotyped (call rate >95 percent), with an average call rate of 99.5 percent. Concordance between the genomic control DNA samples was 100 percent. SNPs with a minor allele frequency of less than 0.01 (n=11) or were not in the Hardy-Weinberg equilibrium (n=5) or were monomorphic (n=8) in this study population were excluded, resulting in 375 SNPs in the analyses.

### Statistical Analysis

Descriptive analysis for study patients: Clinical characteristics of the 248 SCLC patients were first summarized by vital status, and then assessed on their association with survival. Kaplan-Meier curves were obtained for each covariate, and a stepwise selection process using Cox proportional hazards regression was used to identify adjustment variables for the genetic analyses.

Single-SNP Assessments: For each SNP, a Cox regression model was used to assess the associations with survival following lung cancer diagnosis, both before and after adjusting for the five covariates identified above. The primary analysis tested the significance of the association between survival and the ordinal count of the number of minor alleles (0, 1, or 2) carried by each individual. Secondary assessments compared heterozygotes and rare allele homozygotes to the common allele homozygotes to further assess the potential genetic mode of action. From both analyses, hazard ratios (HR), 95% confidence intervals (CI), p-values, and q-values that assessed the probability that a p-value might be false positive were extracted (Storey, J. Royal Statisical Society (B), 64(Part 3):479-498 (2002) and Storey and Tibshirani, PNAS, 100:9440-9445 (2003)).

Whole-Gene Principal Components Analysis: In order to assess whether different analytical approaches resulted in consistent findings, a principal components analysis (PCA) was performed on the candidate genes. Minor allele count variables were used to identify the principal components that captured 95% of the variability in the SNPs for each gene. For the few instances where genotyping data had missing values, the mean genotype value was imputed. Principal components to perform an omnibus test of significance for the association between each of the genes in the glutathione and DNA repair pathways and survival in multivariable Cox proportional hazards regression models were identified. P-values for the global tests were obtained, along with simple summaries of the outcome of the PCA.

Haplotype Analysis: In order to further evaluate genes whose SNPs displayed evidence for association with survival, the associations between haplotypes in selected genes and survival were tested using tools implemented (http://cran.rproject.org/web/packages/haplo.stats/index.html) in the Haplo.Stats package; Schaid et al., Am. J. Human Genetics, 70(2):425-434 (2002)). A Cox proportional hazards regression model was used to test simultaneously the significance of the covariates representing the expected number of each of the candidate haplotypes. Global tests of significance were obtained while adjusting for the same covariates as in the single-SNP analyses. Following the omnibus tests of significance, analyses were performed for each of the haplotypes, estimated HRs, and 95% CIs, as with the single-SNP analyses. All analyses were carried out using SAS (SAS Institute, Inc., Cary, N.C.) and S-Plus (Insightful Corp., Seattle, Wash.) software systems.

### Results

Patient characteristics and clinical prognostic factors for survival are provided in Table 2. Among the 248 genotyped patients, the median follow-up time was 17 months. 64 (26%) were still alive at the closure of this study. A stepwise model selection identified five variables that were potential confounders, i.e., age, sex, pack-years, treatment modality, and stage. They were adjusted in the final model. Table 3 presents summaries of the hazard ratios, 95% confidence intervals, and p-values for these five covariates.

TABLE 2
Demographic and clinical features of the cohort by survival status,

| | Alive | | Dead | | |
|---|---|---|---|---|---|
| Feature | (N = 64) | | (N = 184) | | P-value² |
| Gender | | | | | |
| Male | 32 | (50.0) | 79 | (42.9) | 0.328 |
| Female | 32 | (50.0) | 105 | (57.1) | |
| Mean Age at | 63.7 | (8.9, 43) | 65.0 | (10.3, 27) | |
| Diagnosis (SD, | | | | | |
| minimum) | | | | | |
| Age at Diagnosis | | | | | |
| Age <= 70 | 52 | (81.3) | 125 | (67.9) | 0.042 |
| Age > 70 | 12 | (18.7) | 59 | (32.1) | |
| Mean Pack Years | 61.6 | (32.3) | 59.9 | (32.3) | |
| (SD) | | | | | |
| Pack Years | | | | | |
| <40 Pack Years | 14 | (23.0) | 51 | (29.1) | 0.470 |
| 40-60 Pack Years | 24 | (39.3) | 55 | (31.4) | |
| >60 Pack Years | 23 | (37.7) | 69 | (39.4) | |
| Cigarette Exposure | | | | | |
| Never Smoker | 0 | (0.0) | 1 | (0.5) | 0.899 |
| NS with ETS¹ | 3 | (4.7) | 8 | (4.3) | |
| Light Smoker | 4 | (6.3) | 14 | (7.6) | |
| Moderate Smoker | 12 | (18.8) | 42 | (22.8) | |
| Heavy Smoker | 45 | (70.3) | 119 | (64.7) | |
| Treatment | | | | | |
| Chemo Alone | 8 | (14.0) | 59 | (34.7) | 0.012 |
| Chemo/Surgery | 3 | (5.3) | 8 | (4.7) | |
| Chemo/Radiation | 39 | (68.4) | 95 | (55.9) | |
| Chemo/Radiation/ | 7 | (12.2) | 8 | (4.7) | |
| Surgery | | | | | |
| Stage | | | | | |
| Limited | 52 | (82.5) | 89 | (49.2) | <0.001 |
| Extensive | 11 | (17.5) | 92 | (50.8) | |

Values are presented as a number (percent) unless otherwise indicated.
¹ETS is defined as environmental tobacco exposure.
²Chi-square tests were used for categorical variables, and t-tests were used for continuous variables.

TABLE 3
Results for Cox proportional hazards model for clinical predictors.

| | Hazard | | |
|---|---|---|---|
| Feature | Ratio | 95% CI | P-value |
| Gender | 1.221 | (0.884, 1.687) | 0.226 |
| Stage Limited (Extensive | 0.371 | (0.247, 0.557) | <0.001 |
| as the reference group) | | | |
| Treatment Chemo Alone | 2.980 | (1.008, 8.808) | 0.048 |
| (Chemo/Surgery/Radiation | | | |
| as the reference group) | | | |
| Treatment Chemo/Surgery | 2.370 | (0.710, 7.911) | 0.161 |
| Treatment Chemo/Radiation | 2.343 | (0.849, 6.464) | 0.100 |
| Age at Diagnosis | 1.020 | (1.001, 1.040) | 0.037 |
| Pack Years | 0.998 | (0.993, 1.003) | 0.544 |

Single SNP Analysis Results: Of the 375 SNPs that were assessed for association with survival, 21 (11 genes) had p-values for trend test of less than 0.05, after adjusting for five covariates. Fifteen of the SNPs were from the glutathione pathway and six were from the DNA repair pathway (Table 4). The top three SNPs (rs11597282, rs2025096, and rs7265992) had q-values less than 0.25, suggesting a 1:3 odds of being false positive results. Two of the SNPs were in the GSS gene (rs2025096 and rs7265992), and one was in ABCC2 (rs11597282).

TABLE 4
Significant SNPs after adjusting for clinical variables in single SNP analysis.

| | | | Q | P- | HR | Common | Hetero | Minor |
|---|---|---|---|---|---|---|---|---|
| SNP | Gene | SNP | value¹ | value | (95% CI)² | (%) | (%) | (%) |
| | ABCC1 | rs2239330 | 0.32 | 0.0094 | 0.7 (0.53 0.92) | GG(52.82) | AG(39.11) | AA(8.06) |
| | ABCC2 | rs11597282 | 0.10 | 0.0007 | 4.24 (1.83, 9.80) | GG(95.56) | AG(4.44) | AA(0) |
| | ABCC3 | rs2277624 | 0.43 | 0.048 | 0.75 (0.56, 0.99) | GG(59.27) | AG(35.08) | AA(5.65) |
| | | rs1729775 | 0.31 | 0.0095 | 0.74 (0.59, 0.93) | GG(33.87) | AG(47.18) | AA(18.95) |
| | | rs1189428 | 0.43 | 0.0331 | 0.78 (0.62, 0.98) | GG(23.48) | AG(55.06) | AA(21.46) |
| | ABCC4 | rs11568658 | 0.43 | 0.0344 | 0.42 (0.19, 0.94) | CC(95.16) | AC(4.84) | AA(0) |
| | | rs7993878 | 0.43 | 0.0363 | 1.45 (1.02, 2.04) | GG(76.21) | AG(22.98) | AA(0.81) |
| GSH | | rs17189561 | 0.43 | 0.0435 | 0.71 (0.51, 0.99) | AA(68.55) | AG(29.44) | GG(2.02) |
| | | rs2025096 | 0.10 | 0.0008 | 0.57 (0.41, 0.79) | GG(63.71) | AG(32.66) | AA(3.63) |
| | | rs7265992 | 0.17 | 0.002 | 1.68 (1.21, 2.32) | GG(68.55) | AG(30.24) | AA(1.21) |
| | GSS | rs6060127 | 0.43 | 0.0228 | 1.29 (1.03, 1.61) | GG(45.97) | CG(44.35) | CC(9.68) |
| | | rs2236270 | 0.43 | 0.0337 | 0.76 (0.59, 0.98) | CC(34.68) | AC(54.03) | AA(11.29) |
| | | rs2273684 | 0.43 | 0.0367 | 0.78 (0.62, 0.99) | AA(28.23) | AC(52.82) | CC(18.95) |
| | GSTA3 | rs557135 | 0.43 | 0.0331 | 1.30 (1.02, 1.64) | AA(35.89) | AG(50) | GG(14.11) |
| | GSTT1 | rs11550605 | 0.43 | 0.0257 | 3.94 (1.18, 13.15) | AA(98.56) | AC(1.44) | CC(0) |
| | PARP | rs7988810 | 0.43 | 0.0176 | 1.30 (1.05, 1.61) | AA(8.87) | AG(42.51) | GG(18.62) |
| | | rs7984513 | 0.43 | 0.0285 | 1.29 (1.03, 1.62) | GG(0.77) | AG(48.18) | AA(21.05) |
| DNA | RAD52 | rs10744729 | 0.43 | 0.0311 | 0.78 (0.62, 0.98) | CC(9.44) | AC(52.02) | AA(18.55) |
| | RRM1 | rs1662162 | 0.43 | 0.0305 | 1.57 (1.04, 2.35) | GG(5.48 | AG(14.11) | AA(0.4) |
| | XRCC1 | rs2854510 | 0.43 | 0.0038 | 1.54 (1.15, 2.07) | AA(63.31) | AG(33.47) | GG(3.23) |
| | | rs1001581 | 0.22 | 0.005 | 0.70 (0.54, 0.90) | GG(8.71) | AG(47.98) | AA(13.31) |

Notes:
¹The Q value estimated the false discovery rate for the companion p-value.
²The hazard ratio (HR) was obtained by trend test for each SNP where the ordinal count of the number of minor alleles was used in the Cox model after adjusting for age, gender, tumor stage, treatment, and pack-years of smoking.

Gene-based Analysis Results: Using whole-gene principal components analyses, 3 of the 49 genes were significantly associated with survival and had adjusted p-values of less than 0.05. These genes were the same ones that harbored SNPs with small p-values: GSS, ABCC2, and XRCC1, with adjusted p-values of 0.002, 0.04, and 0.03, respectively.

Haplotype Analyses: Of the three genes significant via principal components analyses, two were significantly associated with survival in the haplotype analyses: ABCC2 (p=0.002) and XRCC1 (0.015). The third, GSS, had a global p-value of 0.095. Four haplotypes in ABCC2 were associated with a lower risk of death (Table 5). For example, the GGGGACGCGGA (SEQ ID NO:1) haplotype was associated with a nearly five-fold lower risk (HR: 0.21, 95% CI: 0.07-0.58), and the AGGGCAAAGGA (SEQ ID NO:2) haplotype was associated with a nearly three-fold lower risk (HR: 0.39, 95% CI: 0.18-0.83). One haplotype in XRCC1, GAACG, was associated with a greater than five-fold risk of death (HR: 5.65, 95% CI: 2.52-12.69).

TABLE 5
Haplotypes significantly associated with survival of SCLC.

| | | | | | 95% |
|---|---|---|---|---|---|
| | | | | Hazard | Confidence |
| Gene | Haplotype | Frequency | p-value | Ratio | Interval |
| ABCC2 | GGGGACGCGGA (SEQ ID NO: 1) | 0.021 | 0.003 | 0.21 | 0.07-0.58 |
| | AAGAACGAGGA (SEQ ID NO: 3) | 0.027 | 0.005 | 0.18 | 0.05-0.59 |
| | AGGGCAAAGGA (SEQ ID NO: 2) | 0.072 | 0.014 | 0.39 | 0.18-0.83 |
| | GGGAACGCGAA (SEQ ID NO: 4) | 0.032 | 0.022 | 0.28 | 0.09-0.84 |
| GSS | AGGAAAC | 0.053 | 0.003 | 3.62 | 1.56-8.39 |
| XRCC1 | GAACG | 0.039 | <0.0001 | 5.65 | 2.52-12.69 |

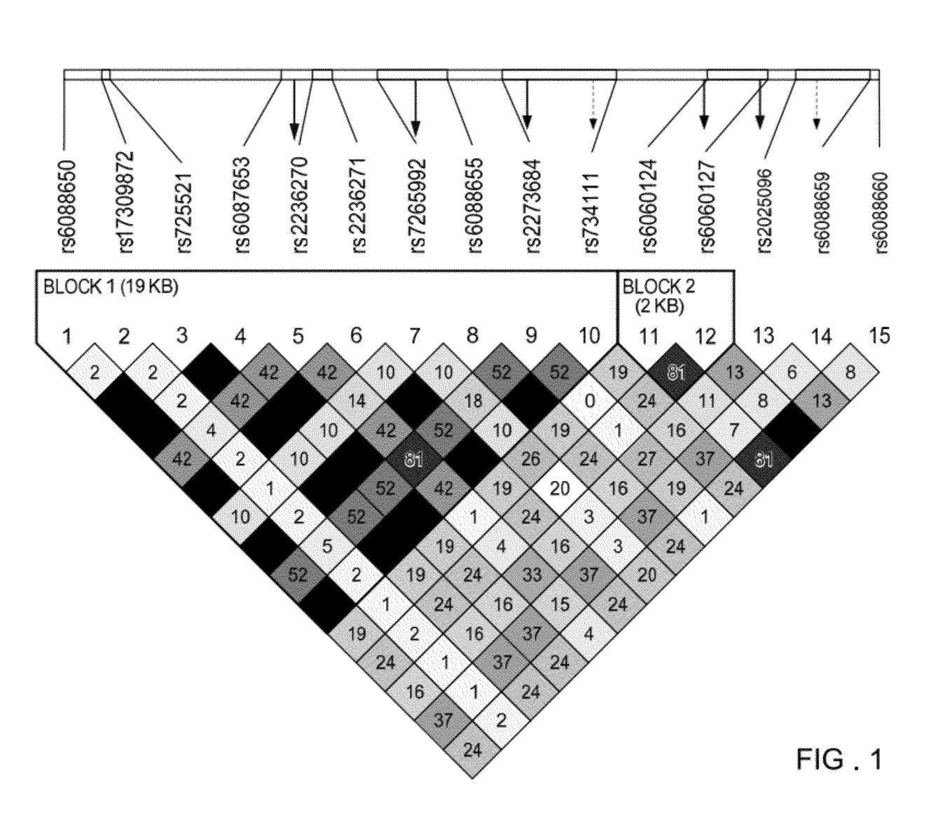

The results provided herein did not demonstrate any significant association for genes such as GSTP1, ERCC1, and ERCC2. The results provided herein demonstrate a significant effect of several tested genes, particularly GSS, ABCC2, and XRCC1. Glutathione synthetase (GSS) participates in the second step glutathione biosynthesis. Among the seven SNPs tested herein, five exhibited significant association with patient survival in single SNP analysis. For example, rs7265992 and rs6060127 were associated with increased risk of death, while rs2025096, rs2236270, and rs2273684 were associated with reduced risk (FIG. 1).

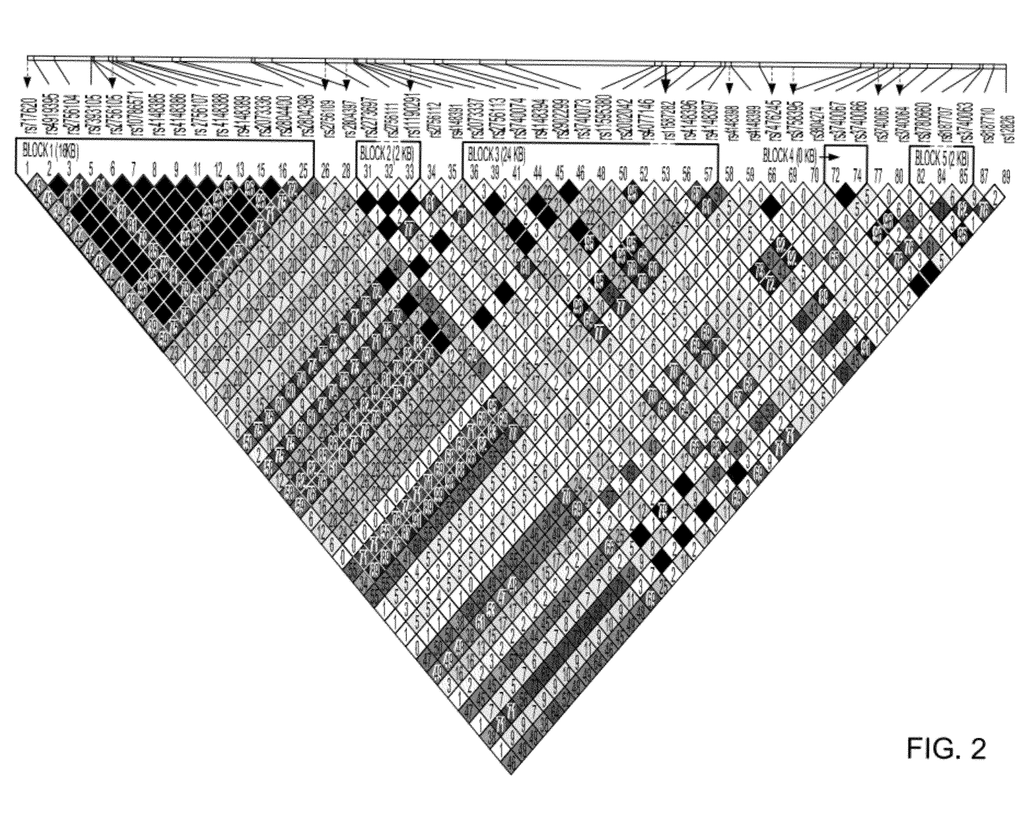

ABCC2 (or MRP2) is a member of the multidrug resistance protein (MRP) family that performs a similar function of transporting glutathione conjugates across the cell membrane. Among the 11 tag-SNPs studied from the ABCC2 gene, rs11597282 (FIG. 2) was found to be significantly associated with survival. Haplotype analysis revealed four haplotypes that were significantly associated with SCLC survival, suggesting certain allele combinations may be more predictive and capture variations that single SNP analysis may have missed, either through true haplotypic effects or by capturing simpler effects marked by these haplotypes.

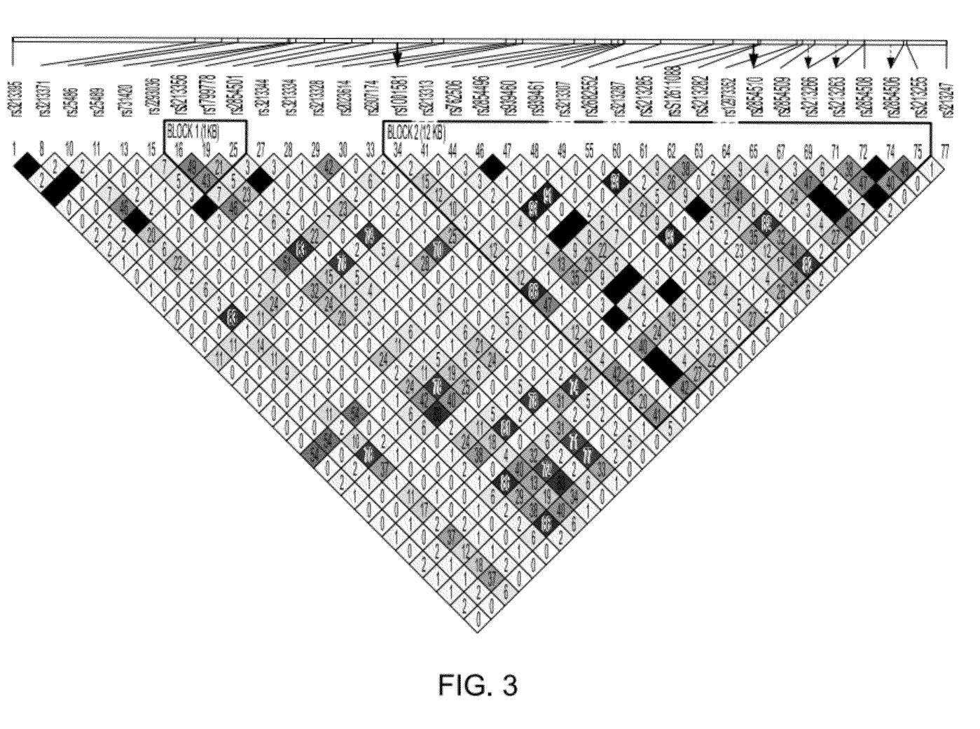

XRCC1 is one of the most common genes studied in the DNA base excision repair pathway. Among the five SNPs in the SCLC data, rs100158 was correlated with improved survival, and rs2854510 was associated with decreased survival. Both SNPs are intronic and are more likely to be the surrogates of other functional variations in the same region with high linkage disequilibrium (FIG. 3).

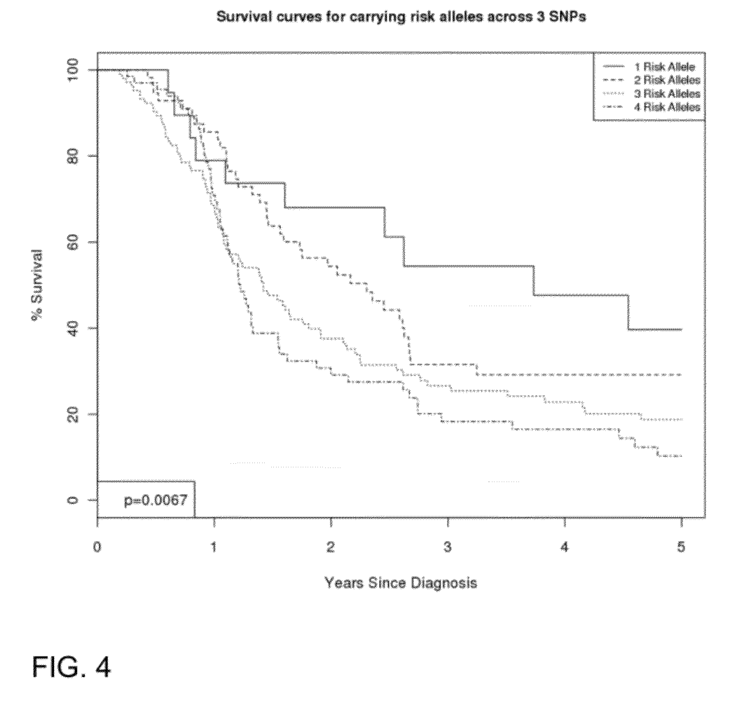

In summary, the results provided herein demonstrate that genetic variations in glutathione metabolic and DNA repair pathways are associated with survival among SCLC patients. Genetic variation of GSS, ABCC2, and XRCC1 were associated with overall survival of SCLC. The associations were significant not only in a single SNP test, but also at the whole gene level and haplotype analysis. The three appear to have an addictive or synergetic effect on treatment response and resistance through modulating the concentration of chemotherapy agents in the cells or their survivability after DNA damage (FIG. 4). The distribution and make-up of the risk allele (RA) groups shown in FIG. 4 are presented in Table 6. These results indicate that targeted genotyping of these genes can be used to stratify patients into good or bad responders to chemo-radiation therapy so that a customized treatment plan can be developed.

TABLE 6
Risk Allele (RA) count per gene.

| # Risk | | Fre- | Per- | Cumulative | Cumulative |
|---|---|---|---|---|---|
| alleles | Risk Alleles | quency | cent | Frequency | Percent |
| 1 | 0 RA in ABCC2 | 14 | 5.65 | 14 | 5.65 |
| | 1 RA in GSS | | | | |
| | 0 RA in XRCC1 | | | | |
| 1 | 0 RA in ABCC2 | 5 | 2.02 | 19 | 7.66 |
| | 0 RA in GSS | | | | |
| | 1 RA in XRCC1 | | | | |
| 2 | 0 RA in ABCC2 | 18 | 7.26 | 37 | 14.92 |
| | 2 RA in GSS | | | | |
| | 0 RA in XRCC1 | | | | |
| 2 | 0 RA in ABCC2 | 36 | 14.52 | 73 | 29.44 |
| | 1 RA in GSS | | | | |
| | 1 RA in XRCC1 | | | | |
| 2 | 0 RA in ABCC2 | 4 | 1.61 | 77 | 31.05 |
| | 0 RA in GSS | | | | |
| | 2 RA in XRCC1 | | | | |
| 2 | 1 RA in ABCC2 | 1 | 0.40 | 78 | 31.45 |
| | 1 RA in GSS | | | | |
| | 0 RA in XRCC1 | | | | |
| 3 | 0 RA in ABCC2 | 75 | 30.24 | 153 | 61.69 |
| | 2 RA in GSS | | | | |
| | 1 RA in XRCC1 | | | | |
| 3 | 0 RA in ABCC2 | 28 | 11.29 | 181 | 72.98 |
| | 1 RA in GSS | | | | |
| | 2 RA in XRCC1 | | | | |
| 3 | 1 RA in ABCC2 | 1 | 0.40 | 182 | 73.39 |
| | 1 RA in GSS | | | | |
| | 1 RA in XRCC1 | | | | |
| 4 | 0 RA in ABCC2 | 57 | 22.98 | 239 | 96.37 |
| | 2 RA in GSS | | | | |
| | 2 RA in XRCC1 | | | | |
| 4* | 1 RA in ABCC2 | 6 | 2.42 | 245 | 98.79 |
| | 2 RA in GSS | | | | |
| | 2 RA in XRCC1 | | | | |
| 4 | 1 RA in ABCC2 | 2 | 0.81 | 247 | 99.60 |
| | 2 RA in GSS | | | | |
| | 1 RA in XRCC1 | | | | |
| 4 | 1 RA in ABCC2 | 1 | 0.40 | 248 | 100.00 |
| | 1 RA in GSS | | | | |
| | 2 RA in XRCC1 | | | | |

*Had 5 risk alleles, but grouped with group that had 4 risk alleles for analysis.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing lung cancer, wherein said method comprises:
(a) performing a nucleic acid sequencing reaction to detect the presence of a genetic variation in ABCC2 nucleic acid,
(b) performing a nucleic acid sequencing reaction to detect the presence of a genetic variation in GSS or XRCC1 nucleic acid, and
(c) classifying said mammal as being susceptible to a poor lung cancer outcome based at least in part on said presence of said genetic variation in ABCC2 nucleic acid and said presence of said genetic variation in GSS or XRCC1 nucleic acid.

2. The method of claim 1, wherein said mammal is a human.

3. The method of claim 1, wherein said genetic variation in ABCC2 nucleic acid is rs11597282.

4. The method of claim 1, wherein said method comprises detecting the presence of a genetic variation in GSS nucleic acid.

5. The method of claim 4, wherein said genetic variation in GSS nucleic acid is rs2025096, rs7265992, or rs6060127.

6. The method of claim 1, wherein said method comprises detecting the presence of a genetic variation in XRCC1 nucleic acid.

7. The method of claim 6, wherein said genetic variation in XRCC1 nucleic acid is rs2854510 or rs1001581.

8. The method of claim 1, wherein said poor lung cancer outcome comprises death within two years of diagnosis of lung cancer.

9. The method of claim 1, wherein said poor lung cancer outcome comprises death within four years of diagnosis of lung cancer.

10. The method of claim 1, wherein said lung cancer is small cell lung cancer.

11. A method for managing patient care for a human having lung cancer and being treated with chemotherapy or radiation therapy, wherein said method comprises:
(a) performing a nucleic acid sequencing reaction to detect the presence of a genetic variation in ABCC2 nucleic acid,
(b) performing a nucleic acid sequencing reaction to detect the presence of a genetic variation in GSS or XRCC1 nucleic acid, and
(c) removing said human from said chemotherapy or said radiation therapy based at least in part on said presence of said genetic variation in ABCC2 nucleic acid and said presence of said genetic variation in GSS or XRCC1 nucleic acid.

12. The method of claim 11, wherein said genetic variation in ABCC2 nucleic acid is rs11597282.

13. The method of claim 11, wherein said method comprises detecting the presence of a genetic variation in GSS nucleic acid.

14. The method of claim 13, wherein said genetic variation in GSS nucleic acid is rs2025096, rs7265992, or rs6060127.

15. The method of claim 11, wherein said method comprises detecting the presence of a genetic variation in XRCC1 nucleic acid.

16. The method of claim 15, wherein said genetic variation in XRCC1 nucleic acid is rs2854510 or rs1001581.

17. The method of claim 11, wherein said lung cancer is small cell lung cancer.
